(19) United States
(12) Patent Application Publication
(10) Pub. No.: US 2026/0165765 A1
(43) Pub. Date: Jun. 18, 2026

(54) CATHETER

(71) Applicant: Japan Lifeline Co., Ltd., Tokyo (JP)

(72) Inventors: Kohei SAKAKI, Tokyo (JP); Makoto KATO, Tokyo (JP); Ryo ISHIDA, Tokyo (JP); Shota FUJII, Tokyo (JP)

(21) Appl. No.: 19/462,709

(22) Filed: Jan. 28, 2026

Related U.S. Application Data
(63) Continuation of application No. PCT/JP2024/018508, filed on May 20, 2024.

(30) Foreign Application Priority Data
Sep. 26, 2023 (JP) 2023-164050

(51) Int. Cl. A61B 18/00

(52) U.S. Cl. CPC A61B 18/00; A61B 2018/0016; A61B 2018/00172; A61B 2018/00184; A61B 2018/00267; A61B 2018/00577

(57) ABSTRACT

A catheter includes a shaft inserted into a body and a plurality of sets of splines 3, each set being composed of two splines connected to a distal end side of the shaft. The two splines 3 constituting a set of splines 3 are arranged so as to sandwich at least one spline 3 belonging to another set. The plurality of sets of splines 3 are each constrained in their movements relative to each other by a single constraining member.

### CROSS REFERENCE TO RELATED APPLICATIONS

This application is a continuation of International Application No. PCT/JP2024/018508, filed on May 20, 2024, which claims the benefit of and priority to Japanese Patent Application 2023-164050, filed on Sep. 26, 2023, the entire contents of which are incorporated herein by reference.

### TECHNICAL FIELD

The present disclosure relates to a catheter.

### BACKGROUND

A catheter is a type of medical device configured to be inserted into the body for diagnosis or treatment. As an example, a catheter provided with a shaft and a basket electrode assembly coupled to a distal end of the shaft has been known (for example, see JP 2016-507349 A). Here, the basket electrode assembly includes a plurality of splines. In addition, the basket electrode assembly is configured to deform from a contracted shape into an expanded shape by deforming the splines.

### SUMMARY

In the catheter described above, when the splines are deformed while in contact with tissue in the body, there is a risk that the spacing between the splines may become too narrow or too wide.

The present disclosure has been made in view of the above circumstances, and its object is to provide a catheter capable of deforming while maintaining appropriate spacing between splines.

One aspect of the present disclosure is a catheter. This catheter includes a shaft configured to be inserted into a body and a plurality of sets of splines, each set being composed of two splines connected to a distal end side of the shaft. The two splines constituting the set of splines are arranged so as to sandwich at least one spline belonging to another set. The plurality of sets of splines are each constrained in their movement relative to each other by a respective single constraining member.

Another aspect of the present disclosure is a catheter. This catheter includes a shaft inserted into the body and a plurality of splines connected to a distal end side of the shaft. The plurality of splines includes a first spline and a second spline. The first spline and the second spline are arranged so as to sandwich at least one other spline and are constrained in their movement relative to each other by a first constraining member.

Any arbitrary combination of the above components, as well as conversions of the expressions of the present disclosure between methods, devices, systems, and the like, are also valid aspects of the present disclosure.

The catheter of the present disclosure can deform while maintaining appropriate spacing between splines.

### BRIEF DESCRIPTION OF DRAWINGS

FIG. 1 is a perspective view schematically showing an example of a deformation state near the distal end of a catheter according to a first embodiment of the present disclosure.

FIG. 2 is a perspective view schematically showing another example of a deformation state near the distal end of a catheter according to the first embodiment of the present disclosure.

FIG. 3 is a perspective view of the catheter shown in FIG. 1 with the splines removed.

FIG. 4 is a perspective view of the catheter shown in FIG. 1 with the distal end member removed.

FIG. 5 is an enlarged view of the distal end region shown in FIG. 4.

FIG. 6 is a side view schematically showing an example of the structure near the distal end of the catheter of the present embodiment.

FIG. 7 is a cross-sectional view along line A-A shown in FIG. 6.

FIG. 8 is a cross-sectional view along line B-B shown in FIG. 6.

FIG. 9 is a schematic diagram showing an example of the overall structure of the catheter of the present disclosure.

FIG. 10 is a cross-sectional view along line C-C shown in FIG. 6.

FIG. 11 is a perspective view schematically showing an example of a deformation state near the distal end of a catheter according to a second embodiment.

FIG. 12 is an enlarged view of the distal end region shown in FIG. 11, with the distal end member removed from the catheter shown in FIG. 11.

FIG. 13 is a schematic view from the distal end side of the catheter shown in FIG. 11, with the distal end member removed.

FIG. 14 is a perspective view schematically showing an example of a deformation state near the distal end of a catheter according to a third embodiment.

FIG. 15 is a diagram showing an enlarged view of the connecting portion of the plurality of constraining members shown in FIG. 14.

FIG. 16 is a diagram showing an example in which the connecting portion of each constraining member shown in FIG. 14 is not exposed from the distal end side of the splines.

### DESCRIPTION OF EMBODIMENTS

Hereinafter, the present disclosure will be described with reference to the drawings based on preferred embodiments. The embodiments are not intended to limit the present disclosure but are illustrative, and all features and combinations thereof described in the embodiments are not necessarily essential to the present disclosure. The same or equivalent components, members, and processes shown in the respective drawings are denoted by the same reference numerals, and repeated explanations are omitted as appropriate. In addition, the scale and shape of each part shown in the drawings are set for convenience to facilitate understanding and are not to be construed as limiting unless specifically stated otherwise. Furthermore, when terms such as “first” and “second” are used in the present specification or claims, such terms do not indicate any order or importance unless otherwise specified, but are used merely to distinguish one component from another. Some members that are not important for explaining the embodiments are omitted from the drawings.

### First Embodiment

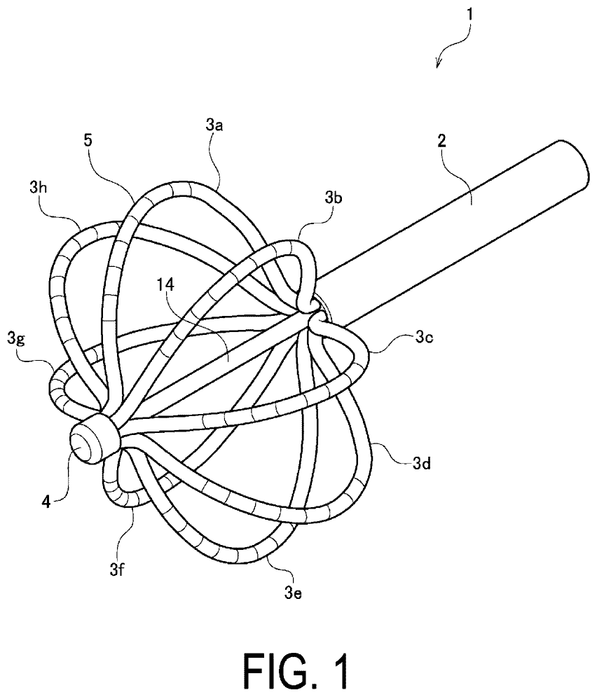

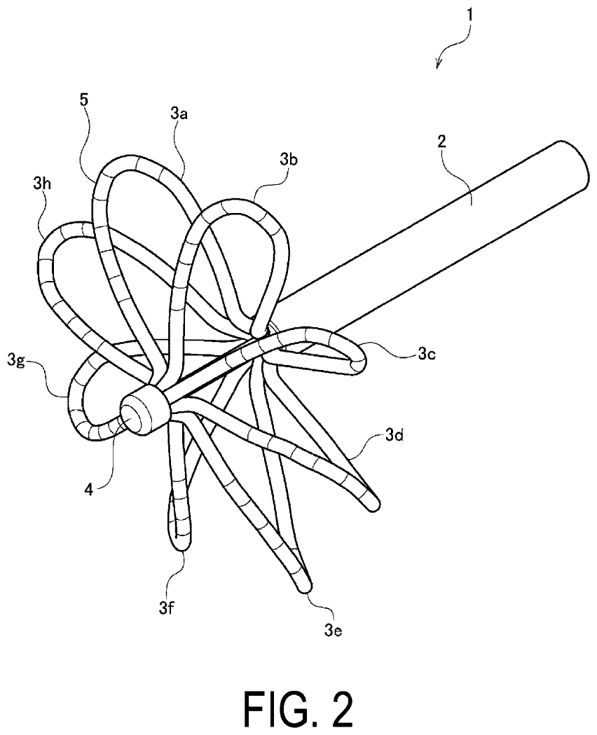

FIG. 1 is a perspective view schematically showing an example of a deformation state near the distal end of a catheter 1 according to the first embodiment of the present disclosure. FIG. 2 is a perspective view schematically showing another example of a deformation state near the distal end of the catheter 1 according to the first embodiment of the present disclosure. As shown in FIGS. 1 and 2, the catheter 1 includes a shaft 2 inserted into the body, a plurality of splines 3a to 3h connected to the distal end side of the shaft 2, and a distal end member 4 connected to the distal end side of each of the plurality of splines 3a to 3h. Hereinafter, descriptions common to the plurality of splines 3a to 3h are simply referred to as spline 3. For other reference numerals with alphabets appended, descriptions common to those reference numerals are described without the alphabets as appropriate. Hereinafter, as appropriate, the side of the catheter 1 inserted into the body is referred to as the “distal end side,” and the side placed outside the body is referred to as the “proximal end side.” For each member constituting the catheter 1, the side corresponding to the distal end side of the catheter 1 is referred to as the “distal end side” of that member, and the side corresponding to the proximal end side of the catheter 1 is referred to as the “proximal end side” of that member.

The shaft 2 may be an elongated circular tubular member. The length of the shaft 2 is, for example, 800 mm to 1800 mm. The outer diameter of the shaft 2 is, for example, 2.0 mm to 5.0 mm. The material constituting the shaft 2 may be a material having flexibility and biocompatibility. For example, the shaft 2 is formed of a known resin such as polyolefin or polyamide.

The spline 3 is a member that connects the shaft 2 and the distal end member 4. The spline 3 may be a circular tubular member like the shaft 2. The length of the spline 3 when extended in a linear manner is, for example, 20 mm to 50 mm. The outer diameter of the spline 3 is, for example, 0.5 mm to 2.0 mm. The material constituting the spline 3 may be a material having flexibility and biocompatibility. For example, the spline 3 is formed of a known resin such as polyolefin or polyamide, like the shaft 2.

The catheter 1 according to the present embodiment includes eight splines 3a to 3h. The splines 3a to 3h are arranged adjacent to each other in this order in a clockwise direction along the circumferential direction when viewed from the distal end side. That is, spline 3a and spline 3b, spline 3b and spline 3c, spline 3c and spline 3d, spline 3d and spline 3e, spline 3e and spline 3f, spline 3f and spline 3g, spline 3g and spline 3h, and spline 3h and spline 3a are respectively adjacent to each other. Additionally, in a plane orthogonal to the central axis of the shaft 2, the splines 3 are arranged spaced apart from each other.

The splines 3 are connected to the shaft 2. For example, a portion (proximal end portion) including a proximal end of the spline 3 is inserted into the distal end side of the shaft 2. The proximal end portion of the spline 3 and the shaft 2 are joined to each other by a known joining method such as welding or bonding with an adhesive.

The spline 3 is configured to change its shape, i.e., deform, in response to a deformation operation described later. Specifically, the shape of each spline 3 changes between a non-deployed shape or contracted shape in which each spline 3 is not deployed along the central axis of the shaft 2 and a deployed shape or expanded shape in which each spline 3 is deployed along the central axis of the shaft 2 from the non-deployed shape. An example of the non-deployed shape may include a “petal shape” (see FIG. 2) as will hereinafter be described in detail. Meanwhile, an example of the deployed shape may include a “basket shape” (see FIG. 1) in which each spline 3 is deployed from the petal shape along the central axis of the shaft 2.

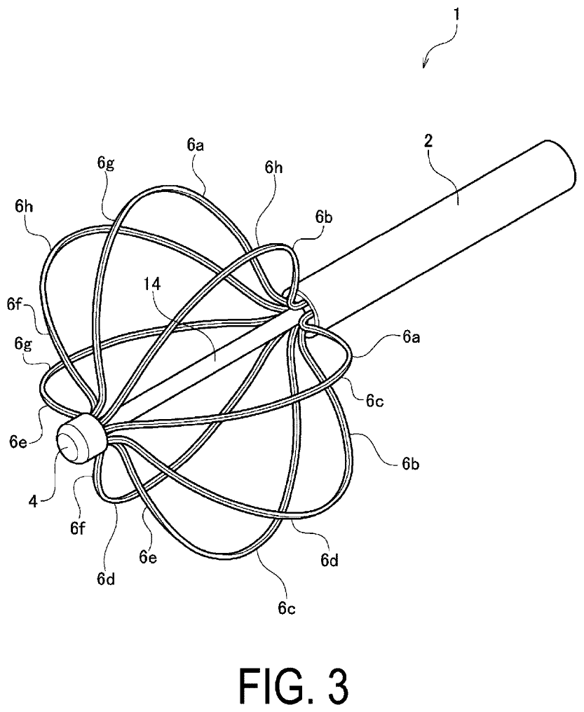

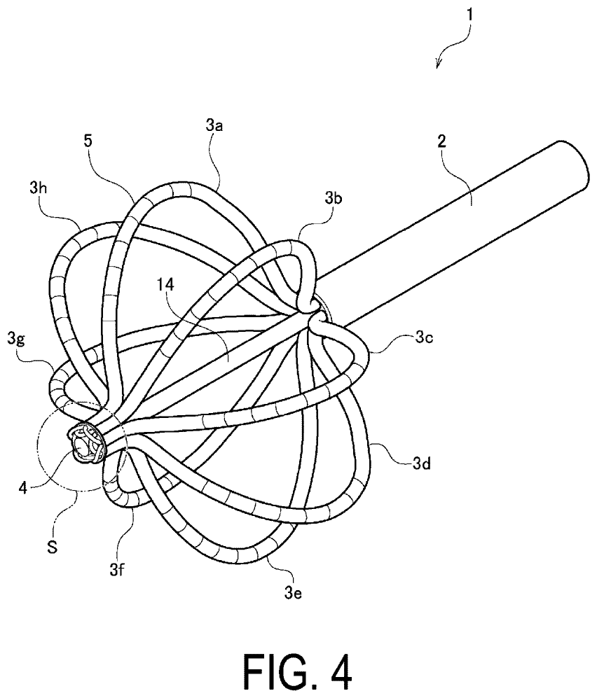

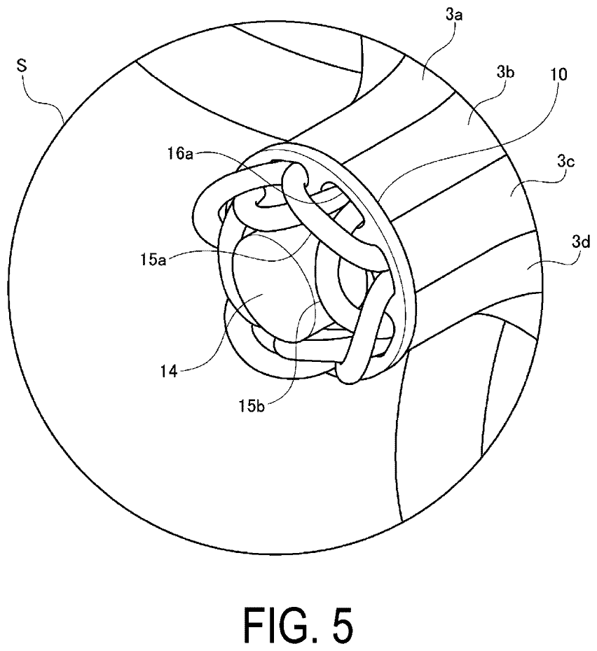

FIG. 3 is a perspective view of members constituting the catheter 1 shown in FIG. 1, with the splines 3 removed. FIG. 4 is a perspective view of members constituting the catheter 1 shown in FIG. 1, with the distal end member 4 removed. FIG. 5 is an enlarged view of the distal end region S shown in FIG. 4. As shown in FIGS. 3 to 5, the spline 3a and the spline 3c are constrained in their movement relative to each other by a constraining member 6a. Here, being constrained in their movement relative to each other means that when the spline 3a and the spline 3c are deformed, they deform while maintaining an appropriate spacing therebetween. The spacing here refers to the shortest distance between the distal end side of the spline 3a and the distal end side of the spline 3c. Therefore, even if a deformation operation is performed while the spline 3a is in contact with tissue in the body, the spline 3a and the spline 3c deform while maintaining an appropriate spacing via the constraining member 6a. Since this configuration is satisfied, the catheter 1 of the present embodiment can deform into a desired shape, i.e., a non-deployed shape or a deployed shape, so as to maintain an appropriate spacing between the splines 3.

The spline 3a and the spline 3c, whose movements relative to each other are constrained by the constraining member 6a, are arranged so as to sandwich the spline 3b and are not adjacent to each other. Therefore, compared to constraining the movements of adjacent splines 3, constraining the movements of non-adjacent splines 3 can make processing easier. As the number of splines 3 included in the catheter 1 increases, more precise processing is required, so the above effect of this configuration becomes more pronounced.

The above configuration can also be expressed as follows. That is, when the spline 3a is designated as a first spline, the spline 3c as a second spline, and the constraining member 6a as a first constraining member, the first spline and the second spline are arranged so as to sandwich at least one other spline and are constrained in their movements relative to each other by the first constraining member.

As described above, the spline 3a and the spline 3c constitute a set of splines 3 whose movements relative to each other are constrained by a single constraining member 6a. Similarly, the spline 3b and the spline 3d constitute another set of splines 3 whose movements relative to each other are constrained by a single constraining member 6b. Additionally, the splines 3a and 3c constituting a set of splines 3 are arranged so as to sandwich one spline 3b belonging to another set. Thus, in the same manner as described above, the catheter 1 of the present embodiment can deform into a desired shape while maintaining appropriate spacing between the splines 3 and can make processing easier.

The above configuration regarding the set of the spline 3a and the spline 3c and the set of the spline 3b and the spline 3d may also apply to other splines 3. That is, splines 3 arranged so as to sandwich at least one spline may each have their movements relative to each other constrained by a single constraining member 6. Specifically, the spline 3c and the spline 3e may have their movements relative to each other constrained by a constraining member 6c. The spline 3d and the spline 3f may have their movements relative to each other constrained by a constraining member 6d. The spline 3e and the spline 3g may have their movements relative to each other constrained by a constraining member 6e. The spline 3f and the spline 3h may have their movements relative to each other constrained by a constraining member 6f. The spline 3g and the spline 3a may have their movements relative to each other constrained by a constraining member 6g. The spline 3h and the spline 3b may have their movements relative to each other constrained by a constraining member 6h. In other words, the catheter 1 includes a plurality of sets of splines 3, each set having their movements relative to each other constrained by a single constraining member 6. Here, one set of splines 3 is composed of two splines 3. As described above, one spline 3 forms different sets with two other splines 3. For example, the spline 3a forms a set with the spline 3c and also forms a set with the spline 3g.

In the present embodiment, an example is shown in which a set of splines 3 is arranged so as to sandwich one spline 3. However, a set of splines 3 may be arranged so as to sandwich at least one other spline 3. That is, a set of splines 3 may be arranged so as to sandwich two splines 3 or sandwich three or more splines 3.

The constraining member 6 may have any shape, structure, or material as long as it is a member capable of constraining the movements of the two splines 3 constituting a set of splines 3 relative to each other. As an example, the constraining member 6 is a wire. The material of the wire may be a shape-memory alloy such as a nickel-titanium alloy.

The constraining member 6 may have a first end and a second end. At least a portion of the first end side of the constraining member 6 may be located within one spline 3 constituting a set of splines 3, and at least a portion of the second end side of the constraining member 6 may be located within the other spline 3 constituting the set of splines 3. The constraining member 6 may have a connecting portion that connects the distal end of one spline 3 and the distal end of the other spline 3. The connecting portion in the present embodiment is a protruding portion 15 that protrudes further toward the distal end side than the distal end of one spline 3 and the distal end of the other spline 3. Here, at least a portion of the first end side refers to a portion of the constraining member 6 having a certain length continuous from the first end side of the protruding portion 15. Also, at least a portion of the second end side refers to a portion of the constraining member 6 having a certain length continuous from the second end side of the protruding portion 15. When the constraining member 6 has a configuration in which the distal ends of a set of splines 3 are connected by the connecting portion, the catheter 1 of the present embodiment connects the distal ends of non-adjacent splines 3, which are farther apart, rather than connecting the distal ends of adjacent splines 3, thereby making processing easier.

For example, at least a portion of the first end side of the constraining member 6a may be located within the spline 3a, and at least a portion of the second end side of the constraining member 6a may be located within the spline 3c. The constraining member 6a may have a protruding portion 15a that protrudes further toward the distal end side than the distal end of the spline 3a and the distal end of the spline 3c.

The first end of the constraining member 6 may be located further toward the proximal end side than the proximal end of one spline 3. The second end of the constraining member 6 may be located further toward the proximal end side than the proximal end of the other spline 3. For example, the first end of the constraining member 6a may be located further toward the proximal end side than the proximal end of the spline 3a. Also, for example, the second end of the constraining member 6a may be located further toward the proximal end side than the proximal end of the spline 3c. If this configuration is satisfied, the constraining member 6 serves as a framework that maintains the shape of the two splines 3 constituting a set of splines 3.

When the constraining member 6 is a wire, the protruding portion 15 may have a U-shaped bent shape. When at least one of the set of splines 3 constrained by the constraining member 6 is deformed, the constraining member 6 also deforms in conjunction. At this time, the stress generated by the change in shape is concentrated in the protruding portion 15. When the protruding portion 15 has a shape bent in a U-shape, the stress described above can be effectively relieved, and even with repeated deformation, the risk of fracture of the protruding portion 15 is reduced.

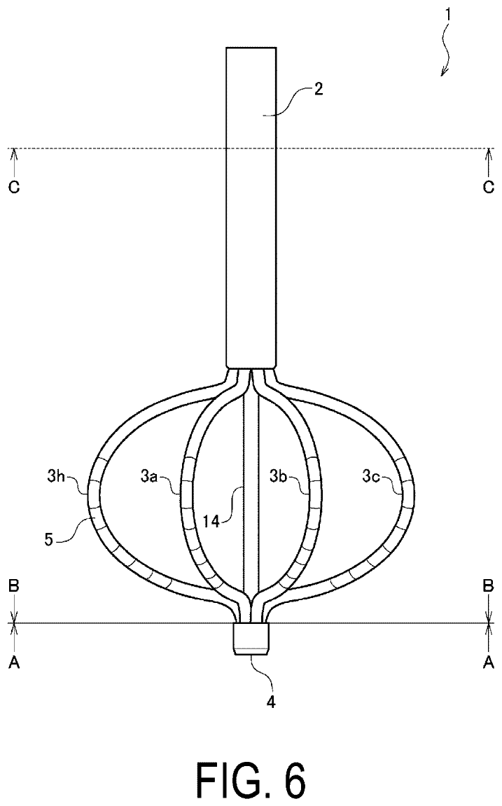

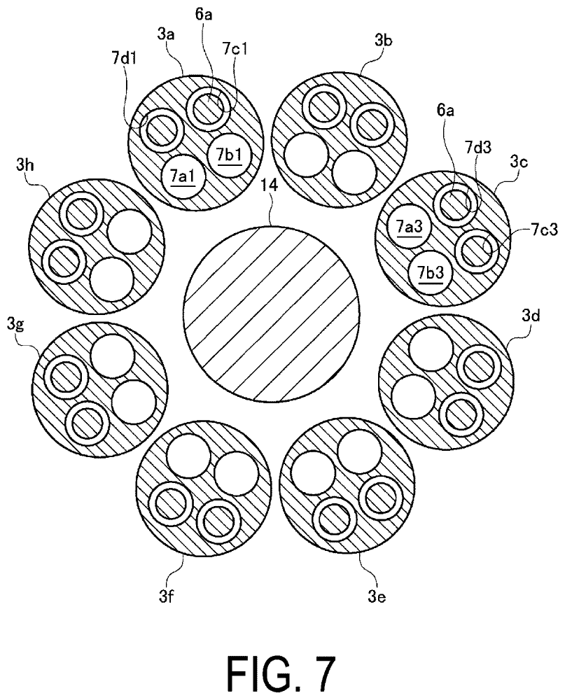

FIG. 6 is a side view schematically showing an example of the structure near the distal end of the catheter 1 of the present embodiment. FIG. 7 is a cross-sectional view along line A-A shown in FIG. 6. As shown in FIG. 7, the spline 3a may have a multi-lumen structure. Specifically, the spline 3a includes four lumens 7 (7a1, 7b1, 7c1, 7d1) extending from the distal end to the proximal end along a central axis of the spline 3a. Here, the lumens 7a1, 7b1 are located closer to the central axis of the shaft 2 than the lumens 7c1, 7d1 are. The lumens 7b1, 7c1 are located closer to the spline 3b than the lumens 7a1, 7d1 are.

The spline 3c may have a multi-lumen structure similar to the spline 3a. Specifically, the spline 3c has four lumens 7 (7a3, 7b3, 7c3, 7d3) extending from the distal end to the proximal end along the central axis of the spline 3c. Here, the lumens 7a3 and 7b3 are located closer to the central axis of the shaft 2 compared to the lumens 7c3 and 7d3. Also, the lumens 7a3 and 7d3 are located closer to the spline 3b compared to the lumens 7b3 and 7c3.

The first end side of the constraining member 6a may be inserted into the lumen 7c1 of the spline 3a. On the other hand, the second end side of the constraining member 6a may be inserted into the lumen 7d3 of the spline 3c. Thus, by inserting the constraining member 6a into the lumens 7c1 and 7d3, which are located away from the central axis of the shaft 2 and are the closest to each other among the lumens 7 of the spline 3a and the spline 3c, the spline 3a and the spline 3c are maintained so as to have a more appropriate spacing.

In the above-described embodiment, the spline 3a, the spline 3c, and the constraining member 6a were used as an example for explanation, but the same may apply to other splines 3 and constraining members 6. In this case, since the constraining member 6 is inserted into a set of lumens 7 that are located away from the central axis of the shaft 2 and are the closest to each other for each set of splines 3, the constraining members 6 can be arranged less likely to interfere with each other.

Each spline 3 may include one or more electrodes 5 as shown in FIG. 6. The electrode 5 is a ring-shaped electrode for potential measurement or ablation. The electrodes 5 included in the same spline 3 are disposed separated from each other along the longitudinal direction of the spline 3. In this case, the intervals between the adjacent electrodes 5 may be uniform or may be different. The number of electrodes 5 included in each of the splines 3 may be the same or may be different.

The electrode 5 is formed of a material having conductivity. For example, the electrode 5 is formed of a metal having good electrical conductivity, such as aluminum (Al), copper (Cu), stainless steel, gold (Au), or platinum (Pt). The length of the electrode 5 is, for example, 0.5 mm to 2.0 mm. The outer diameter of the electrode 5 may be equivalent to the outer diameter of the spline 3, and is, for example, 0.5 mm to 1.6 mm.

A conductive wire is individually and electrically connected to each of the electrodes 5. The conductive wire extends from inside the spline 3 through the shaft 2 and into the handle 8 described below to be connected via the handle 8 to an external power supply device. As an example, each conductive wire connected to each electrode 5 of the spline 3a passes through the lumen 7a1 or the lumen 7b1 of the spline 3a in a state of being electrically insulated from each other. The same applies to each spline 3 other than the spline 3a.

As shown in FIGS. 2 and 4, the distal end member 4 may cover a portion including the distal end (hereinafter referred to as the “distal end portion”) of the spline 3a, the distal end portion of the spline 3c, and the protruding portion 15a of the constraining member 6a. In other words, the distal end portion of the spline 3a, the distal end portion of the spline 3c, and the protruding portion 15a of the constraining member 6a may be covered by the distal end member 4. The distal end member 4 may have any shape, and has a cap shape, for example. The distal end member 4 may be formed of any material, and is formed of a known resin, such as nylon, nylon elastomer, or polycarbonate, or a known metal, such as stainless steel. Note that the distal end member 4 may cover all of the distal end portions of the splines 3 and the protruding portions 15 of the constraining members 6.

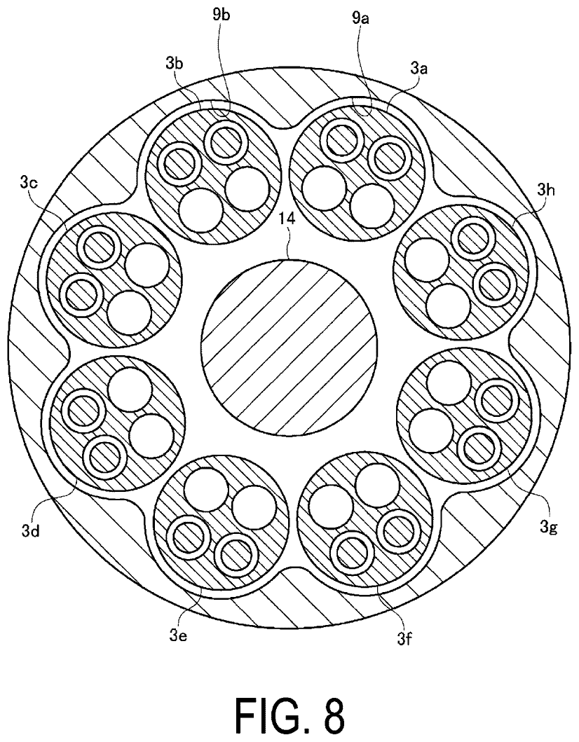

FIG. 8 is a cross-sectional view along line B-B shown in FIG. 6. As shown in FIG. 8, in a plan view from the proximal end side of the distal end member 4, the wall portion constituting the inner surface of the distal end member 4 may have adjacent recesses 9a and 9b. At least a portion of the distal end portion of the spline 3a may be located within the recess 9a, and at least a portion of the distal end portion of the spline 3b may be located within the recess 9b. Here, at least a portion of the distal end portion of the spline 3 being located within the recess 9 means that, in a plan view from the proximal end side of the distal end member 4, the spline 3 has a portion overlapping the region of the recess 9. If this configuration is satisfied, the recesses 9a and 9b can prevent the positions of the distal end portion of the spline 3a and the distal end portion of the spline 3b from shifting when the spline 3a and the spline 3b are deformed.

As an example, the shapes of the recess 9a and the recess 9b are semicircular in a plan view from the proximal end side of the distal end member 4. The wall portion constituting the recess 9a and the spline 3a may or may not be in contact. Similarly, the wall portion constituting the recess 9b and the spline 3b may or may not be in contact. FIG. 8 shows an example in which the wall portion constituting the inner surface of the distal end member 4 has eight recesses 9. Thus, the wall portion constituting the inner surface of the distal end member 4 may have the recesses 9 corresponding to the respective splines 3.

The interior of the distal end member 4 may be filled with an adhesive. If this configuration is satisfied, the spline 3a, the spline 3c, and the constraining member 6a are each firmly joined by the adhesive, so the spline 3a and the spline 3c are maintained so as to have a more appropriate spacing. This also applies to splines 3 other than the spline 3a and the spline 3c and to constraining members 6 other than the constraining member 6a.

As shown in FIG. 5, the distal end member 4 may include a bundling member 10 inside that bundles the protruding portion 15a of the constraining member 6a. Here, bundling the protruding portion 15a means constraining the protruding portion 15a such that the protruding portion 15a does not move in a direction along a plane orthogonal to the central axis of the shaft 2. If this configuration is satisfied, the bundling member 10 suppresses twisting of the constraining member 6a, thereby making it easier to effectively synchronize the deformation of the spline 3a and the spline 3c via the constraining member 6a. Note that the bundling member 10 may bundle the protruding portions 15 of all of the constraining members 6.

The bundling member 10 may be of any shape, structure, and material as long as the bundling member 10 can bundle the protruding portion 15a. As an example, the shape of the bundling member 10 is plate-shaped, having an upper surface on the distal end side (hereinafter referred to as the “first surface”) and a lower surface on the proximal end side, which is the back surface of the first surface (hereinafter referred to as the “second surface”). The second surface faces the distal end surface of the spline 3a and the distal end surface of the spline 3c. Furthermore, the bundling member 10 is provided with a through-hole 16a, which penetrates from the first surface to the second surface, into which the protruding portion 15a of the constraining member 6a is inserted. The side surface connecting the first surface and the second surface of the bundling member 10 (hereinafter referred to as the “third surface”) may have a shape corresponding to the wall portion constituting the inner surface of the distal end member 4. Specifically, it may have a shape having protrusions corresponding to the respective recesses 9 of the distal end member 4. The material constituting the bundling member 10 may be a material having high rigidity. For example, the bundling member 10 is formed of a known metal such as stainless steel. Note that the bundling member 10 may be provided with through-holes 16 corresponding to the protruding portions 15 of the respective constraining members 6.

The through-hole 16a may have any shape as long as the protruding portion 15a of the constraining member 6a can be inserted. For example, the shape of the through-hole 16a is an elliptical shape in plan view seen from the first surface side of the bundling member 10. Note that the same may apply to the other through-holes 16.

As shown in FIG. 5, the constraining member 6a corresponding to the spline 3a and the spline 3c and the constraining member 6b corresponding to the spline 3b and the spline 3d may have their respective protruding portions 15a and 15b bent in different directions when viewed from the distal end side. Here, “viewed from the distal end side” means viewing the catheter 1 from a position further toward the distal end side than the catheter 1. The constraining member 6a and the constraining member 6b are in a relationship where they partially overlap in the circumferential direction of the catheter 1. In the present embodiment, the constraining member 6a is bent toward the radially outer side of the catheter 1 compared to the constraining member 6b. The same may apply to other constraining members 6 that partially overlap in the circumferential direction of the catheter 1, excluding the constraining member 6a and the constraining member 6b.

Thus, the first constraining member 6 corresponding to the first set of splines 3 and the second constraining member 6 corresponding to the second set of splines 3, which includes a spline 3 sandwiched between the two splines 3 constituting the first set of splines 3, may have their respective protruding portions 15 bent in different directions when viewed from the distal end side. This makes it less likely for the constraining members 6 to interfere with each other, thereby making processing easier. Such constraining members 6 can be formed, for example, by wires of a shape-memory alloy bent into different shapes.

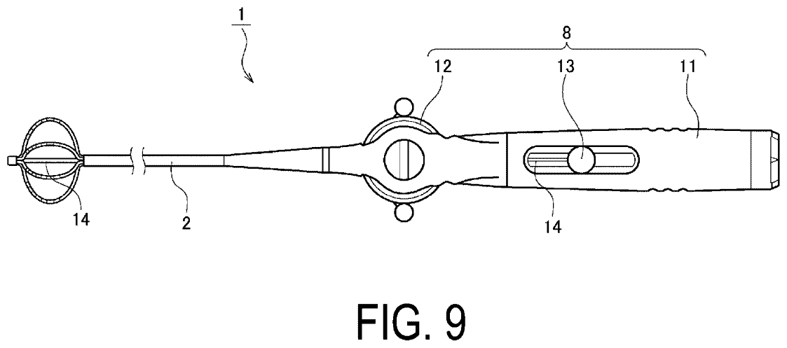

FIG. 9 is a schematic diagram showing an example of the overall structure of the catheter 1 of the present disclosure. As shown in FIG. 9, the catheter 1 may include a handle 8 connected to the proximal end side of the shaft 2. The handle 8 is a portion that an operator, such as a doctor, holds or grips during use of the catheter 1. The handle 8 may include a handle body 11 mounted on the proximal end side of the shaft 2, a rotational operation portion 12, and a slide member 13.

The handle body 11 corresponds to a portion that is actually held by the operator. The handle body 11 may have any shape. The handle body 11 has a shape extending along the central axis of the shaft 2, for example. In addition, the handle body 11 is formed of, for example, a known resin such as polycarbonate, polyacetal, or ABS.

The rotational operation portion 12 is a portion operated, such as by rotation, during an operation to bend or flex near the distal end of the shaft 2 in opposite directions. Proximal ends of a pair of pull wires are fixed to the rotational operation portion 12 in the handle body 11. Distal ends of the pair of pull wires pass through the handle body 11 and the shaft 2 to be fixed to the distal end side of the shaft 2. Therefore, when the rotational operation portion 12 is operated, the pull wires are pulled toward the proximal end side, and the distal end side of the shaft 2, to which the distal ends of the pull wires are fixed, bends or flexes.

The slide member 13 is a portion operated by the operator, such as by sliding, when deforming the shape of the spline 3 between the aforementioned non-deployed shape or contracted shape and the deployed shape or expanded shape. The slide member 13 is slidable along the central axis of the shaft 2 in the handle body 11.

A proximal end of a deformation member 14 is fixed to the slide member 13. A distal end of the deformation member 14 passes through the shaft 2 from the inside of the handle body 11 to be fixed to the inside of the distal end member 4. The slide member 13 is movable to any position in the handle body 11 along the central axis of the shaft 2. Therefore, depending on the position of the slide member 13, the shape of the spline 3 can be deformed into the aforementioned non-deployed shape or contracted shape, the deployed shape or expanded shape, or any intermediate shape between the non-deployed shape and the deployed shape.

The deformation member 14 may be of any shape, structure, and material as long as the deformation member 14 is an elongated member. For example, the deformation member 14 is a wire thicker than the constraining member 6.

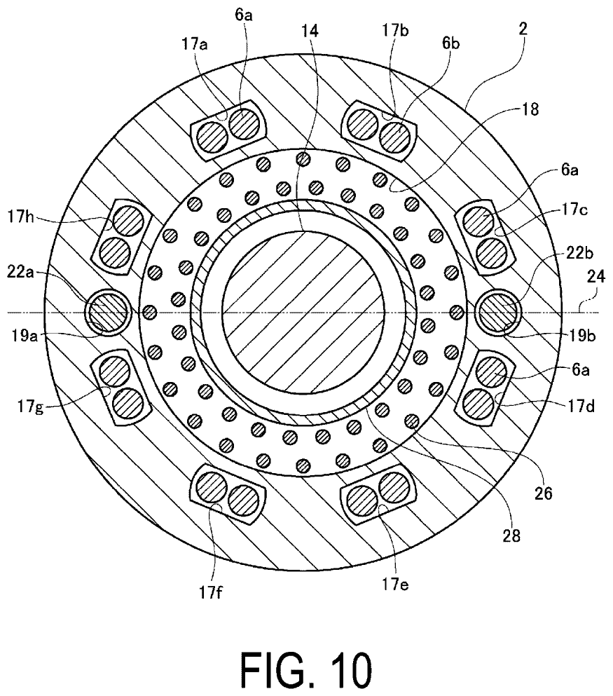

FIG. 10 is a cross-sectional view along line C-C shown in FIG. 6. As shown in FIG. 10, the shaft 2 has a large lumen 18 at a position near the radial center. The large lumen 18 extends from the proximal end to the distal end of the shaft 2. The shaft 2 has eight small lumens 17a to 17h arranged along the circumferential direction around the large lumen 18 and two pull wire lumens 19a and 19b arranged symmetrically with respect to the approximate center of the shaft 2. The eight small lumens 17a to 17h are arranged adjacent to each other in this order in a clockwise direction along the circumferential direction when viewed from the distal end side.

The first end and the second end of each of the plurality of constraining members 6 may be disposed within the shaft 2. Specifically, the first end of the constraining member 6a is disposed within the small lumen 17a, and the second end of the constraining member 6a is disposed within the small lumen 17c. Similarly, the first end of the constraining member 6b is disposed within the small lumen 17b, and the second end of the constraining member 6b is disposed within the small lumen 17d. The same may apply to other constraining members 6.

A pair of pull wires 22a and 22b extend from the proximal end to the distal end within the shaft 2. Specifically, the pull wire 22a is disposed within the pull wire lumen 19a. The pull wire 22b is disposed within the pull wire lumen 19b. Thus, the pair of pull wires 22a and 22b are disposed symmetrically with respect to the approximate center of the shaft 2 in a cross-sectional view orthogonal to the axis of the shaft 2, as shown in FIG. 10.

In the cross-sectional view shown in FIG. 10, the eight small lumens 17a to 17h are arranged line-symmetrically with respect to a straight line 24 passing through the center positions of the pair of pull wires 22a and 22b. Specifically, the small lumen 17a and the small lumen 17f, the small lumen 17b and the small lumen 17e, the small lumen 17c and the small lumen 17d, and the small lumen 17g and the small lumen 17h are each arranged line-symmetrically with respect to the straight line 24. Therefore, the plurality of constraining members 6 disposed within the plurality of small lumens 17 are also arranged line-symmetrically with respect to the straight line 24. This reduces the bias in flexibility or weight balance on both sides of the plane when attempting to bend near the distal end of the shaft 2 in two directions within the same plane using the pair of pull wires 22a and 22b, thereby improving planarity.

In the present embodiment, the term “line-symmetric” is used for convenience, but it is not required that each small lumen 17 be strictly arranged line-symmetrically in the actual shaft 2. For example, each small lumen 17 may be disposed within a predetermined distance from the line-symmetric position. Specifically, the distance of each small lumen 17 from the line-symmetric position may be within 30% of the diameter of the respective small lumen 17, preferably within 20%, and more preferably within 10%.

In the cross-sectional view shown in FIG. 10, the eight small lumens 17a to 17h are arranged point-symmetrically with respect to the axis of the shaft 2. Specifically, the small lumen 17a and the small lumen 17e, the small lumen 17b and the small lumen 17f, the small lumen 17c and the small lumen 17g, and the small lumen 17d and the small lumen 17h are each arranged point-symmetrically with respect to the axis of the shaft 2. Therefore, the plurality of constraining members 6 disposed within the plurality of small lumens 17 are also arranged point-symmetrically with respect to the axis of the shaft 2. This reduces the bias in flexibility or weight balance at positions opposing the axis of the shaft 2, thereby further improving planarity.

In the present embodiment, the term “point-symmetric” is used for convenience, but it is not required that each small lumen 17 be strictly arranged point-symmetrically in the actual shaft 2. For example, each small lumen 17 may be disposed within a predetermined distance from the point-symmetric position. Specifically, the distance of each small lumen 17 from the point-symmetric position may be within 30% of the diameter of the respective small lumen 17, preferably within 20%, and more preferably within 10%.

As described above, by disposing the plurality of constraining members 6 within the small lumens 17 arranged around the large lumen 18 of the shaft 2, a large cross-sectional area orthogonal to the axial direction of the large lumen 18 can be secured. This makes it easier to arrange a plurality of conductive wires 26, an irrigation tube 28, a deformation member 14 disposed at a position near the center within the irrigation tube 28, and the like, which are disposed within the large lumen 18, as shown in FIG. 10.

As described above, the catheter 1 according to the first embodiment has been described as including eight splines 3a to 3h. However, the number of splines 3 included in the catheter 1 is not limited to eight and may be four or more. However, as described above, from the perspective of performing operations with a pair of pull wires 22 in a balanced manner, it is preferable that the number of splines 3 be an even number.

### Second Embodiment

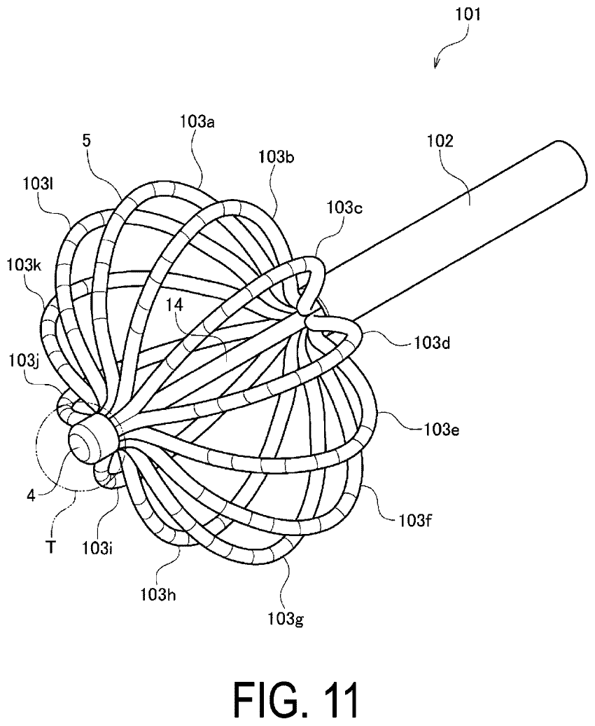

The second embodiment of the present disclosure will be described with reference to FIGS. 11 to 13. In the subsequent embodiment, among the components described in the first embodiment, the same details as in the first embodiment may be applied to the components that are not described below. FIG. 11 is a perspective view schematically showing an example of a deformation state near the distal end of a catheter 101 according to the second embodiment.

The catheter 101 includes a shaft 102 inserted into the body, a plurality of splines 103a to 103l connected to the distal end side of the shaft 102, and a distal end member 104 connected to the distal end side of each of the plurality of splines 103a to 103l. The splines 103a to 103l are arranged adjacent to each other in this order in a clockwise direction along the circumferential direction when viewed from the distal end side. The catheter 101 of the present embodiment differs from the catheter 1 of the first embodiment in that it includes twelve splines 103a to 103l. The configuration of each spline 103 is the same as that of the spline 3 of the first embodiment. The shaft 102 and the distal end member 104 are the same as the shaft 2 and the distal end member 4 of the first embodiment, respectively, except that they are shaped to correspond to the twelve splines 103a to 103l. FIG. 11 shows, similarly to FIG. 1 of the first embodiment, the splines 103 in a “basket shape” as a deployed shape.

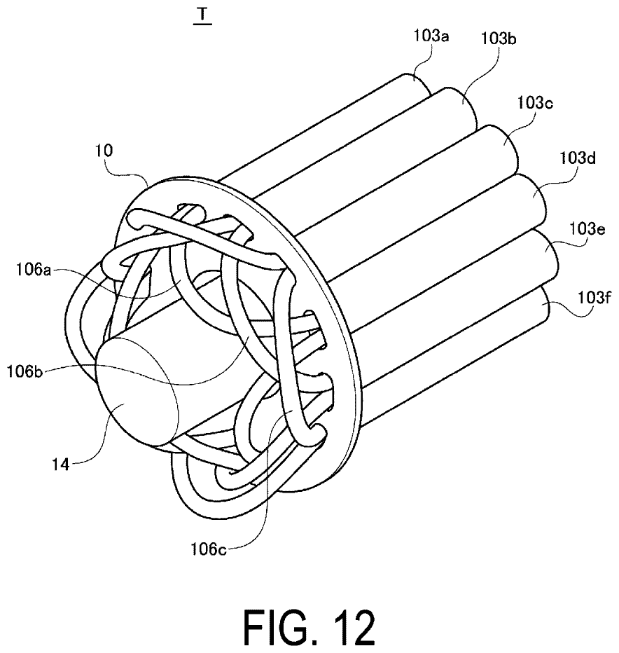

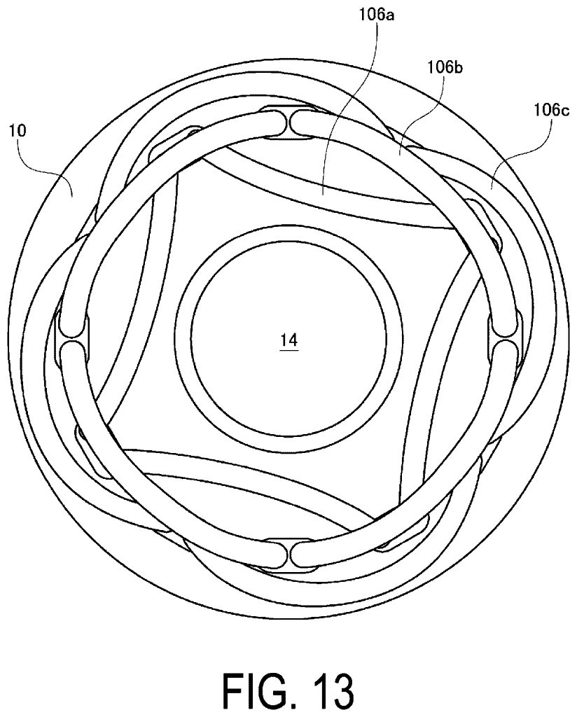

FIG. 12 is an enlarged view of the distal end region T shown in FIG. 11, with the distal end member 104 removed from the components constituting the catheter 101 shown in FIG. 11. FIG. 13 is a schematic view from the distal end side of the catheter 101 shown in FIG. 11, with the distal end member 104 removed. As shown in FIGS. 12 and 13, the constraining member 106a constrains the spline 103a and the spline 103d. The constraining member 106b constrains the spline 103b and the spline 103e. The constraining member 106c constrains the spline 103c and the spline 103f. Thus, in the present embodiment, a set of splines 103a and 103d are arranged so as to sandwich two splines 103b and 103c, each belonging to another set. This applies similarly to other splines 103.

As shown in FIG. 13, the constraining member 106a corresponding to the spline 103a and the spline 103d, the constraining member 106b corresponding to the spline 103b and the spline 103e, and the constraining member 106c corresponding to the spline 103c and the spline 103f may have their respective protruding portions bent in different directions when viewed from the distal end side. The constraining member 106a, the constraining member 106b, and the constraining member 106c are in a relationship where they partially overlap in the circumferential direction of the catheter 101. In the present embodiment, the constraining member 106a is bent toward the radially inner side of the catheter 101 compared to the constraining member 106b and the constraining member 106c. The constraining member 106b has less bending compared to the constraining member 106a and the constraining member 106c.

The constraining member 106c is bent toward the radially outer side of the catheter 101 compared to the constraining member 106a and the constraining member 106b. The same may apply to other constraining members 106 that partially overlap in the circumferential direction of the catheter 101, excluding the constraining member 106a, the constraining member 106b, and the constraining member 106c. Thus, the catheter 101 of the present embodiment can achieve the same effects as those of the first embodiment.

### Third Embodiment

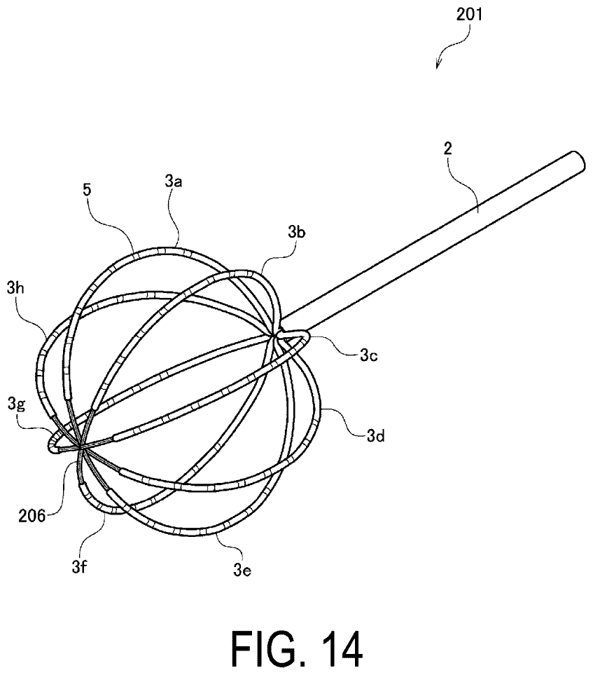

The third embodiment of the present disclosure will be described with reference to FIGS. 14 to 16. FIG. 14 is a perspective view schematically showing an example of a deformation state near the distal end of a catheter 201 according to the third embodiment. The catheter 201 includes a shaft 2 and a plurality of splines 3a to 3h connected to the distal end side of the shaft 2. Although not shown in FIG. 14, the catheter 201 may include a distal end member, similarly to the catheter 1 of the first embodiment.

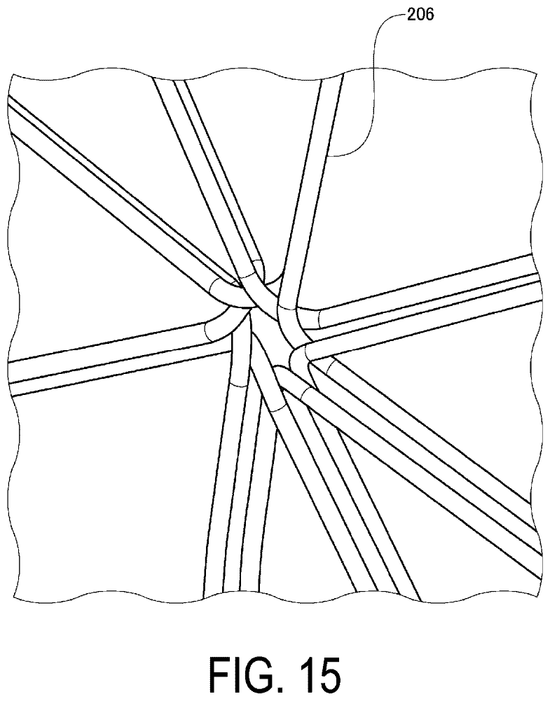

The catheter 201 of the present embodiment differs from the catheter 1 of the first embodiment in that the connecting portion of the constraining member 206 does not protrude toward the distal end side. FIG. 15 is a diagram showing an enlarged view of the connecting portions of a plurality of constraining members 206. As shown in FIG. 15, each constraining member 206 has a structure intertwined with the constraining member 206 adjacent in the circumferential direction. Specifically, in the example shown in FIG. 15, when the clockwise direction in the circumferential direction viewed from the distal end side is defined as the positive direction, the connecting portion of each constraining member 206 is arranged to be positioned further toward the distal end side than the connecting portion of the constraining member 206 adjacent in the negative direction and further toward the proximal end side than the connecting portion of the constraining member 206 adjacent in the positive direction. By having the connecting portions of each constraining member 206 intertwined in this manner, the relative positions of the plurality of constraining members 206 can be stabilized without using any member such as the bundling member 10 of the first embodiment.

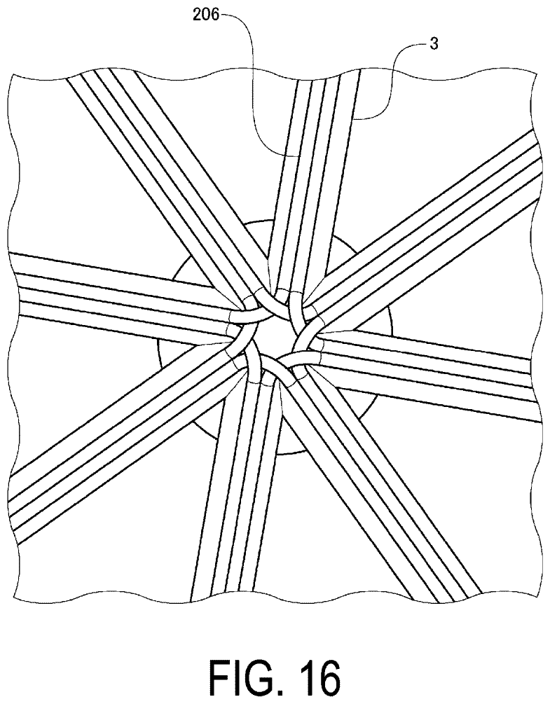

FIG. 16 is a diagram showing an example in which the connecting portion of each constraining member 206 is not exposed from the distal end side of the spline 3. Even with such a configuration, each constraining member 206 can achieve the same effects as described above.

The embodiments of the present disclosure have been described in detail. The above-described embodiments are merely specific examples for implementing the present disclosure. The contents of the embodiments do not limit the technical scope of the present disclosure, and various design modifications such as changes, additions, or deletions of components are possible without departing from the spirit and scope of the present disclosure as defined in the claims. A newly modified embodiment with design changes incorporates the respective effects of the combined embodiments and modifications. In the above-described embodiments, expressions such as “of the present embodiment” or “in the present embodiment” are used to emphasize the contents where such design modifications are possible, but design modifications are also permissible for contents without such expressions. Any arbitrary combination of components included in the respective embodiments is also a valid aspect of the present disclosure. The hatching indicated in the cross-sections of the drawings does not limit the material of the hatched object.

The embodiments may also be specified by the items described below.

### Item 1

A catheter (1) comprising:
a shaft (2) configured to be inserted into a body, and
a plurality of sets of splines (3) connected to a distal end side of the shaft (2),
wherein the two splines (3) constituting the set of splines (3) are arranged so as to sandwich at least one spline (3) belonging to another set, and
the plurality of sets of splines (3) are each constrained in their movements relative to each other by a single constraining member (6).

### Item 2

The catheter (1) according to Item 1, wherein:
the constraining member (6) has a first end and a second end,
at least a portion of the first end side of the constraining member (6) is located within one spline (3) constituting a set of splines (3),
at least a portion of the second end side of the constraining member (6) is located within the other spline (3) constituting the set of splines (3), and
the constraining member (6) has a connecting portion that connects the distal end of one spline (3) and the distal end of the other spline (3).

### Item 3

The catheter (1) according to Item 2, wherein:
the connecting portion of the constraining member (6) is a protruding portion (15) that protrudes further toward the distal end side than the distal end of one spline (3) and the distal end of the other spline (3).

### Item 4

The catheter (1) according to Item 3, wherein:
the first constraining member (6) corresponding to the first set of splines (3) and the second constraining member (6) corresponding to the second set of splines (3), which include a spline (3) sandwiched between the two splines (3) constituting the first set of splines (3), have their respective protruding portions (15) bent in different directions when viewed from the distal end side.

### Item 5

The catheter (1) according to any one of Items 2 to 4, wherein:
the first end and the second end of each of a plurality of the constraining members (6) are disposed within the shaft (2), and
in a cross-sectional view orthogonal to an axis of the shaft (2), the plurality of constraining members (6) are arranged line-symmetrically with respect to a straight line (24) passing through center positions of a pair of pull wires (22).

### Item 6

The catheter (1) according to any one of Items 2 to 4, wherein:
the first end and the second end of each of the plurality of constraining members (6) are disposed within the shaft (2), and
in a cross-sectional view orthogonal to an axis of the shaft (2), the plurality of constraining members (6) are arranged point-symmetrically with respect to the axis of the shaft (2).

### Item 7

The catheter (1) according to Item 3 or Item 4, further comprising:
a distal end member (4) covering distal end portions of each of the plurality of sets of splines (3) and the protruding portions (15) of each of the plurality of constraining members (6).

### Item 8

The catheter (1) according to Item 7, wherein:
the distal end member (4) includes a bundling member (10) inside that bundles the protruding portions (15) of each of the plurality of constraining members (6).

### Item 9

The catheter (1) according to Item 8, wherein:
the bundling member (10) has a plate shape having a first surface and a second surface that is the back surface of the first surface,
the second surface faces the distal end surface of each of the plurality of sets of splines (3), and
the bundling member (10) is provided with a through-hole (16) that extends from the first surface to the second surface and into which the protruding portion (15) of each of the plurality of constraining members (6) is inserted.

### Item 10

The catheter (1) according to any one of Items 1 to 9, wherein:
a plurality of the constraining members are wires.

### Item 11

### A Catheter (1) Comprising:

a shaft (2) configured to be inserted into a body, and
a plurality of splines (3) connected to a distal end side of the shaft (2),
wherein the plurality of splines (3) include a first spline (3) and a second spline (3), and
the first spline (3) and the second spline (3) are arranged so as to sandwich at least one other spline (3) and are constrained in their movements relative to each other by a first constraining member (6).

### INDUSTRIAL APPLICABILITY

The present disclosure can be utilized in a catheter.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. A catheter comprising:
a shaft configured to be inserted into a body; and
a plurality of sets of splines, each set being composed of two splines connected to a distal end side of the shaft; wherein
the two splines constituting the set of splines are arranged so as to sandwich at least one spline belonging to another set, and
the plurality of sets of splines are each constrained in their movements relative to each other by a single constraining member.

2. The catheter according to claim 1, wherein:
the constraining member has a first end and a second end,
at least a portion of the first end side of the constraining member is located within one spline constituting the set of splines,
at least a portion of the second end side of the constraining member is located within the other spline constituting the set of splines, and
the constraining member has a connecting portion that connects the distal end of the one spline and the distal end of the other spline.

3. The catheter according to claim 2, wherein:
the connecting portion of the constraining member is a protruding portion that protrudes further toward the distal end side than the distal end of the one spline and the distal end of the other spline.

4. The catheter according to claim 3, wherein:
a first constraining member corresponding to a first set of splines and a second constraining member corresponding to a second set of splines, which include a spline sandwiched between the two splines constituting the first set of splines, have their respective protruding portions bent in different directions when viewed from the distal end side.

5. The catheter according to claim 2, wherein:
the first end and the second end of each of a plurality of the constraining members are disposed within the shaft, and
in a cross-sectional view orthogonal to an axis of the shaft, the plurality of constraining members are arranged line-symmetrically with respect to a straight line passing through center positions of a pair of pull wires.

6. The catheter according to claim 2, wherein:
the first end and the second end of each of the plurality of constraining members are disposed within the shaft, and
in a cross-sectional view orthogonal to an axis of the shaft, the plurality of constraining members are arranged point-symmetrically with respect to the axis of the shaft.

7. The catheter according to claim 3, further comprising:
a distal end member covering distal end portions of each of the plurality of sets of splines and the protruding portions of each of the plurality of constraining members.

8. The catheter according to claim 7, wherein:
the distal end member includes a bundling member inside that bundles the protruding portions of each of the plurality of constraining members.

9. The catheter according to claim 8, wherein:
the bundling member has a plate shape having a first surface and a second surface that is a back surface of the first surface,
the second surface faces the distal end surface of each of the plurality of sets of splines, and
the bundling member is provided with a through-hole that extends from the first surface to the second surface and into which the protruding portion of each of the plurality of constraining members is inserted.

10. The catheter according to claim 1, wherein:
a plurality of the constraining members are wires.

11. A catheter comprising:
a shaft configured to be inserted into a body, and
a plurality of splines connected to a distal end side of the shaft, wherein
the plurality of splines include a first spline and a second spline, and
the first spline and the second spline are arranged so as to sandwich at least one other spline and are constrained in their movements relative to each other by a first constraining member.
